(19) United States
(12) Patent Application Publication
(10) Pub. No.: US 2005/0071016 A1
(43) Pub. Date: Mar. 31, 2005

(54) Medical metal implants that can be decomposed by corrosion

(76) Inventors: Gerd Hausdorf, Burgwedel (DE); Jacqueline Hausdorf, Burgwedel (DE); Matthias Niemeyer, Hannover (DE); Matthias Peuster, Hannover (DE); Bernd Heublein, Hannover (DE); Phan-Tan Tai, Hannover (DE); Jorg Meyer, Hamburg (DE)

Correspondence Address: Robert W Becker & Associates, Suite B, 707 Highway 66 East, Tijeras, NM, 87059, US

(21) Appl. No.: 10/250,784

(22) PCT Filed: Jan. 5, 2001
(86) PCT No.: PCT/EP01/00085

(51) Int. Cl. A61F 2/02; A61F 2/28

(52) U.S. Cl. 623/23.75; 623/23.71; 606/77; 623/23.53

(57) ABSTRACT

An in vivo, decomposable medical implant is provided and comprises a metal material that contains, as a main alloying constituent, tungsten or a metal from the group rhenium, osmium, and molybdenum. The method for decomposing the implant, via corrosion in a bio system, includes the step of changing the pH level of the bio system, at least at the site of the implant, from a corrosion-inhibiting level to a corrosion-promoting level.

The present invention relates to a medical implant that can be decomposed in vivo comprising a metal material and belonging to the group of implants in accordance with the preamble of claim 1.

Such an implant is known from DE 19731021. In the case of these implants practically at the same time as the implantation a corrosive action is started, which after a certain time leads to the fact that the implant firstly becomes mechanically unstable and then is completely decomposed. With these implants the material is to be selected in such a way that the corrosion proceeds slowly in order to ensure mechanical stability is maintained to the necessary extent. This also results in correspondingly slow decomposition of the implant material, after this implant has fulfilled its function. In practice complete decomposition will need many times the period, for which the mechanical function should continue to remain in place.

Furthermore a process is known for producing so-called coils as vessel sealing systems from a tungsten alloy, which can corrode. Precisely in the case of vessel sealing systems is decomposition of the implant not desirable, in particular if the implantation has not resulted in the vessel closure aimed for.

The object therefore of the present invention is to generally improve an implant from the group specified in the preamble of claim 1 so that the mechanical stability of the implant remains in place for as long a period as necessary and after the mechanical function has been fulfilled; corrosion enables the decomposition of the implant to be accelerated. Another object of the present invention is to produce an active substance depot, which allows the active substance to be released in a deliberately controllable way.

This object is achieved by an invention with the features of claim 1. Another object of the present invention is to provide a process for the decomposition of a metal implant, which enables the rate of the corrosive decomposition of the implant to be controlled in a purposeful manner. This object is achieved by a process with the features of claim 7.

Because it is proposed in the case of the implant embodying the invention that the material contains tungsten as the main alloy or a metal from the group including rhenium, osmium, molybdenum, the implant in the biological environment, for the use of which it is intended, will exhibit corrosion behavior dependent on the pH level, whereby the transition from a non-corrosive condition to a corrosive condition occurs at a pH level, which can be tolerated by the respective biological system. In particular the transition to the corrosive condition is influenced by a process controlling the pH level in the biological system.

In the case of the active substance depot the release of the active substance is assisted by the change in the pH level from the corrosion-inhibiting condition to the corrosion-promoting condition.

With the process according to the invention decomposition of the metal implant is induced as a result of the pH level of the bio system being changed at least at the place of the implant from the corrosion-inhibiting level to a corrosion-promoting level.

Secondary constituents in this case can be a multiplicity of elements, which may also have no influence on the corrosion behavior. With the implant it is advantageous however if the material as the secondary constituent contains one or more elements from the group of lanthanides, in particular cerium, actinides, iron, osmium, tantalum, rhenium, gadolinium, yttrium or scandium. With these alloying elements good corrosion behavior can be achieved for the intended purpose desired. In this case typical compositions are formed so that the main alloying constituent represents more than 75%, in particular 95% to 99.5%, of the material and the remainder to reach 100% is formed from the at least one secondary constituent. Particularly fast decomposition within a certain pH range is possible if the material exhibits a crystalline structure with a particle size of 0.5 μm to 30 μm, in particular 0.5 μm to 5 μm. Then extensive corrosion takes place. However with particle sizes of 10 μm or more inter-crystalline corrosion can also take place, which leads to formation of particles, whereby the body can exude these particles.

In addition it is advantageous if the implant contains metal or non-metal inclusions having the nature of sintered metal, which comprise essentially pure alkali or alkaline earth metal, except the alloy material. These inclusions can promote deliberate corrosion in regard to both the start and rate of corrosion. In addition alkali or alkaline earth ions released as a result of corrosion may become physiologically effective in an advantageous way.

There results an embodiment advantageous in regard to mechanical stability with good corrosion if the implant has an essentially tubular base.

With the process according to the invention the object of changing the pH level of the bio system at least in the place of the implant from the corrosion-inhibiting level to a corrosion-promoting level is achieved. As a result after the implant has fulfilled its mechanical function, fast corrosion can be influenced in a concerted way.

In this case advantageously the pH level of the bio system within the vicinity of the cardiovascular system can be increased to a pH level of 7.4 or higher, preferably to a pH level of more than 7.5 and in particular more than 7.6.

Likewise it can be advantageous if the pH level of the bio system within the vicinity of the urine or bile system is changed, whereby in the urine system for example the pH level can be raised to over 9 or reduced to levels below 7.

The change in the pH level for the promotion of corrosion is advantageously achieved if alkalizing or acidifying substances are supplied to or taken away from the bio system, in particular ascorbic acid, sodium bicarbonate, citrate and/or diuretics (for example frusemide, thiazide, carboanhydrase inhibitors).

An advantageous embodiment of the present process proposes that the pH level of the bio system is changed by supplying or stopping drugs alkalizing the bio system, in particular loop diuretics.

An embodiment of the present invention is described below.

A cardiovascular stent is manufactured with a tubular base from tungsten or a tungsten alloy in the presently known way. The stent is introduced into a restricted blood vessel and is expanded in the region of the vessel restriction. The stent remains there until the vessel has regained sufficient natural stability. Up to this point the pH level in the blood of the patient is maintained at a level of <7.4 by regular administration of ascorbic acid. As soon as it is decided that the support function of the stent is no longer needed, administration of ascorbic acid is stopped and the blood of the patient is alkalized to a pH level of above 7.4 by administering diuretics. In the changed environment the stent will corrode fairly quickly. Relatively fast decomposition of the material results, whereby the material disposed in the blood vessel leads to fast removal of the tungsten particles or tungsten ions arising and thus prevents local build up of any toxic concentration.

The material used in this embodiment is an alloy consisting of 99.2% tungsten and 0.8% cerium with a particle size of approximately 1 μm. In this case extensive corrosion, the decomposition rate of which at a pH level of 7.2 amounts to 20 μm per annum, results in the human bloodstream. By increasing the pH level to 7.4 the decomposition rate rises to 50 μm per annum.

In the case of a second embodiment an active substance depot is produced from a tungsten alloy, whereby active substances materials having the nature of sintered metal with therapeutically effective characteristics (metal ions, drugs, mRNA, vectors) are introduced into the alloy material. The implant is disposed in a position of the bio system, which can be treated outside the bloodstream.

As in the previous embodiment the bio system is firstly kept at a relatively low pH level by administering ascorbic acid or similar active substances.

As soon as the active substances are needed, an alkalizing substance is administered so that the pH level rises. The initial corrosion releases the therapeutically effective material and since it is disposed outside the bloodstream, leads to high local concentration of active substance, which is therapeutically effective without impairing the rest of the bio system. In this way tumors, vessel restrictions can be fought by intima proliferation, other vessel reactions such as fibrosis, but also infections or similar can be fought by concerted selectable local and systemic active substance dosages.

The same applies to implants in the urinary tracts or bile ducts, whereby for controlling the active substance release in the case of these applications a broader pH spectrum is available. Here it is proposed according to a further embodiment that a urinary tract stent is made from an alloy consisting of 98.5% molybdenum and 1.5% tantalum. This stent is stable at a pH level of more than 2, while by changing the pH level to below 2 the corrosive decomposition is accelerated. Apart from tantalum platinum and gold are also possible here as secondary constituents.

As in the first embodiment surgical clips, metal sutures or the like can also be maintained in place until they have fulfilled their function. Afterwards the corrosion and thus decomposition of the material can be induced by deliberately changing the pH level.

1-11. (cancelled)

12. An in vivo, decomposable medical implant from the group including:
stents (coronary stents, peripHeral stents, tracheal stents, bile duct stents, esopHagus stents), surgical clips, osteosynthesis material, biological matrix (foam), metal wiring, metal threads, active substance depots, comprising a metal material,
wherein said material contains, as a main alloying constituent, tungsten or a metal selected from the group consisting of rhenium, osmium and molybdenum.

13. A medical implant according to claim 12, wherein said material contains, as a secondary constituent, at least one element selected from the group consisting of lanthanides, actinides, iron, osmium, tantalum, platinum, gold, rhenium, gadolinium, yttrium and scandium.

14. A medical implant according to claim 13, wherein said lanthanide is cerium.

15. A medical implant according to claim 12, wherein said main alloying constituent represents more than 75% of said material, with any remainder, to form 100%, being formed by at least one secondary constituent.

16. A medical implant according to claim 15, wherein said main alloying constituent represents 95 to 99.5% of said material.

17. A medical implant according to claim 12, wherein said material has a crystalline structure having a particle size of 0.5 to 30 μm.

18. A medical implant according to claim 17, wherein said particle size is 0.5 to 5 μm.

19. A medical implant according to claim 12, wherein said implant, with the exception of said material, contains metal or non-metal inclusions that comprise an essentially pure alkali or alkaline earth metal, a drug, mRNA or a vector.

20. A medical implant according to claim 12, wherein said implant has an essentially tubular base.

21. A method for decomposition of the implant of claim 12 via corrosion in a bio system, including the step of:
changing the pH level of the bio system, at least at a site of the implant, from a corrosion-inhibiting level to a corrosion-promoting level.

22. A method according to claim 21, wherein within the vicinity of a cardiovascular system, the pH level of said bio system is changed to a level of at least 7.4.

23. A method according to claim 22, wherein the pH level of said bio system is changed to a level of at least 7.5.

24. A method according to claim 22, wherein the pH level of said bio system is changed to a level of at least 7.6.

25. A method according to claim 21, wherein within the vicinity of a urine or bio system, the pH level of said bio system is changed from a lower pH level to a higher pH level.

26. A method according to claim 21, wherein the pH level of said bio system is changed by supplying or stopping alkalizing or acidifying substances.

27. A method according to claim 26, wherein said alkalizing or acidifying substances are at least one of the group consisting of ascorbic acid, sodium bicarbonate, citrates, and diuretics.

28. A method according to claim 21, wherein the pH level of said bio system is changed by supplying or stopping drugs that alkalize said bio system.

29. A method according to claim 28, wherein said drugs are loop diuretics.
